(19) United States
(12) Patent Application Publication
(10) Pub. No.: US 2007/0027536 A1
(43) Pub. Date: Feb. 1, 2007

(54) Aortic Valve Annuloplasty Rings

(76) Inventors: Tomislav Mihaljevic, Gates Mills, OH (US); R. Saeid Farivar, Brookline, MA (US); Lawrence H. Cohn, Brookline, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/422,210

(22) Filed: Jun. 5, 2006

Related U.S. Application Data
(63) Continuation of application No. PCT/US04/40517, filed on Dec. 3, 2004.
(60) Provisional application No. 60/526,887, filed on Dec. 4, 2003.

(51) Int. Cl. A61F 2/24

(52) U.S. Cl. 623/2.37

(57) ABSTRACT

An aortic annuloplasty ring may include a ring, having a “C” shape. The ring may be so sized as to fit around and circumferentially engage an aortic root. The ring may be formed at least in part of a biocompatible material so nonresiliently deformable as to permit manual adjustment of the ring. An aortic annuloplasty method may include disposing an aortic annuloplasty ring around an aorta root, the ring having a “C” shape, and the ring being so sized as to fit around and circumferentially engage the aortic root; and deforming the ring to circumferentially engage the aortic root.

### CROSS REFERENCE TO RELATED APPLICATION

This application is a continuation of International Application No. PCT/US2004/040517, filed Dec. 3, 2004, which claims the benefit of U.S. Provisional Application Ser. No. 60/526,887, filed Dec. 4, 2003. The entire contents of these applications are hereby incorporated herein by reference.

### FIELD

The disclosed systems and methods relate generally to systems and methods for aortic valve annuloplasty. More specifically, the disclosed systems and methods relate to annuloplasty rings and methods for deploying annuloplasty rings.

### BACKGROUND

The aortic valve is situated at the junction of the left ventricle of the heart and the root of the aorta. The valve opens to admit blood ejected from the contracting heart into the ascending aorta, and closes to prevent regurgitation of the ejected blood back into the left ventricle. The valve opens and closes by the motion of its constituent leaflets, of which there are typically three (but occasionally two or, rarely, one). When the valve is functioning properly, the leaflets seal the valve by touching one another, referred to as “co-aption” or “coaption.”

A number of pathologic conditions, however, may prevent the perfect coaption of the leaflets. The two broad categories of pathology include disorders of the leaflets themselves and disorders of the fibrous skeletal ring (“annulus”) that supports the leaflets. Leaflet disorders include scarring, fibrosis, and calcification resulting from infection (rheumatic fever), hypertension, or congenital malformation. The resulting thickening or encrustation limits the leaflets' range of motion so that they cannot fully close. Blood is then able to leak through the imperfectly coapted leaflets.

Disorders of the annulus of the aortic valve may result from inherent defects in the annulus or from stretching caused by aortic dilation. Inherent defects may result from trauma to the annulus or from genetic disorders of connective tissue. Dilation of the aorta may result from a wide variety of etiologies, including trauma, genetic disorders (Marfan syndrome and Ehlers-Danlos syndrome), congenital malformation (coarctation of the aorta), infectious disease (syphilis and mycotic infections), inflammatory disorders (rheumatoid arthritis, Takayasu's arteritis), hypertension, and atherosclerosis. When the annulus is deformed, the value leaflets may not touch, even when fully closed.

Currently, aortic valve performance is restored by replacing the valve leaflets and the annulus with a prosthetic structure. The prosthetic structure may be a biomaterial (such as a porcine valve, a human cadaveric valve, or pericardial tissue) or a metallic implant (such as a pyrolite carbon bileaflet valve). Replacement of the aortic valve is a complex procedure necessitating cardiopulmonary bypass and its attendant risks.

### SUMMARY

The present disclosure provides systems and methods for restoring proper coaption of the aortic valve leaflets without subjecting a patient to valve replacement surgery. The inventors have found that the leaflets can be repositioned for proper coaption by engaging a ring around the aortic root, in a subcoronary position, to constrict the root. The applied compression may counteract the distortion of the stretched annulus. The compression can significantly ameliorate the effects of the underlying pathology and delay the need for a valve replacement. In some circumstances, compression can eliminate the need for valve replacement entirely.

In one embodiment, an aortic annuloplasty ring includes a ring, having a “C” shape and being so sized as to fit around and circumferentially engage an aortic root. The ring is formed at least in part of a biocompatible material so deformable as to permit manual adjustment of the ring but stiff enough to keep the shape into which it is adjusted.

In another embodiment, an aortic annuloplasty ring includes a collar having first and second ends that together form a fastener operable to secure the first and second ends together. The collar is thereby so shaped as to engage the aorta circumferentially. The ring further includes a flap depending from the collar for wrapping over the aorta, to prevent distal aneurismal changes. The ring is sized to fit around the aorta, and is transitionable between a first state, in which the fastener does not secure the first and second ends together, and a second state, in which the fastener so secures the first end to the second end that the collar is shaped to engage the aorta circumferentially.

In yet another embodiment, an aortic annuloplasty method includes disposing an aortic annuloplasty ring around the aortic root, and deforming the ring to circumferentially engage it. The ring has a “C” shape and is so sized as to fit around and circumferentially engage the aortic root, formed at least in part of a biocompatible material so deformable as to permit manual adjustment of the ring, and so nonresilient as to keep the shape into which it is deformed against blood pressure or the heart beat's force.

In still another embodiment, an aortic annuloplasty method includes disposing an aortic annuloplasty ring around an aorta, the ring including a collar having first and second ends, the first and second ends forming a fastener operable to secure the first and second ends together, the ring further including a flap depending from the collar; fastening the first and second ends of the collar, thereby so shaping the collar as to engage the aorta circumferentially; and wrapping the flap over the aorta.

### BRIEF DESCRIPTION OF THE DRAWINGS

FIG. 1 depicts an exemplary embodiment of an aortic annuloplasty ring, the ring lying flat.

FIG. 2 depicts an exemplary embodiment of an aortic annuloplasty ring, the ring having a substantially circular shape.

FIG. 3 is a plan view of an exemplary embodiment of an aortic annuloplasty ring having a “C” shape.

FIG. 4 is a perspective view of the ring shown in FIG. 3.

FIGS. 5-9 depict exemplary cross sections taken at line 5-5 of FIG. 3.

FIG. 10 depicts an exemplary embodiment of a ring having a groove.

FIG. 10A depicts an exemplary embodiment of a ring having more than one groove.

FIG. 11 depicts an exemplary embodiment of the deployment of a grooved ring.

FIGS. 12-14 depict exemplary modifications of ring ends.

FIGS. 15-18 depict exemplary ring adjustment systems.

FIGS. 19-20 depict exemplary ring sealing systems.

### DETAILED DESCRIPTION

The disclosed systems and methods facilitate aortic annuloplasty by providing aortic annuloplasty rings that are deployed around the aorta to improve coaption of the aortic valve leaflets.

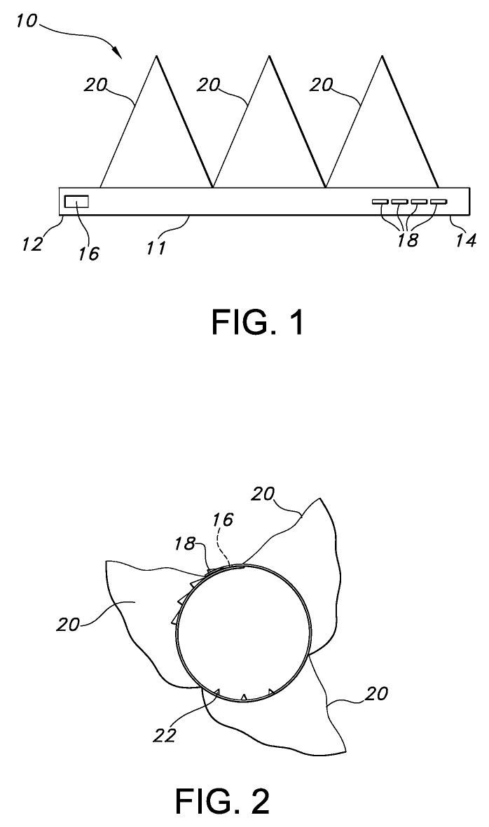

FIG. 1 shows one exemplary embodiment of such a ring. The depicted ring 10 includes a collar 11 having a first end 12 and a second end 14 that cooperate to form a fastener that secures the ends to each other. In the FIG. 1 embodiment, for example, the collar's first end removably and adjustably receives catches 18 on the collar's second end. The ring may be reversibly transitionable between a first state, shown in FIG. 1, in which the two ends are not secured, and the fastener and the collar 11 can lie substantially flat, and a second state, shown in FIG. 2, in which the fastener secures the collar's ends in an endless configuration. Although the FIG. 1 embodiment includes a plurality of catches 18 to make the ring adjustable, some embodiments may instead be fixed in size.

FIG. 2 depicts the ring in its second state, in which the fastener secures the ring 10 in its endless configuration. The second state may be substantially circular, but in any event it will tend to conform to the outer shape of the aorta in the vicinity of the aortic valve so as to engage the aorta circumferentially. FIG. 2 shows an aperture 16 receiving one particular catch 18, but the ring may be adjusted to make the aperture receive a different catch 18. As FIGS. 1 and 2 show, the catches 18 have respective inclined surfaces on one side to facilitate further tightening of the ring, but the opposite-side surfaces impede loosening of the ring; the catches act as a ratcheting mechanism. That is, the aperture 16 may have to be lifted out of contact with the catch 18 to permit loosening. Such an arrangement may be selected both for convenience and for safety. A ring with a preferential adjustment for tightening may improve deployment of the device by preventing the ring from slipping while the operator is fine-tuning its fit. Furthermore, a ring that resists loosening tends to keep its preferred shape and size and is less likely to need its fit revised after initial deployment.

In other embodiments, the catches 18 may be so shaped as to resist adjust in both directions, such as by having ends that are both raised from the surface of the collar 11. In one embodiment, the catches 18 fit lock-and-key with the aperture 16. Such an arrangement can facilitate precise adjustment of the ring during deployment and can also impede undesired tightening of the ring after deployment. Such tightening might otherwise occur, for example, if the ring is tugged by scar tissue.

In other embodiments, the catch 18 may facilitate continuous adjustment, as opposed to the illustrated discrete adjustment. For example, one of the collar's ends may form a slot, and a clamp that slides along the slot and affixes to the collar at a desired position may be attached to the collar's other end.

The ring shown in FIG. 1 includes three flaps 20 that depend from the collar 11 and can be wrapped over the aorta to prevent dilation of the aorta distal to the ring. Other embodiments may have more or fewer flaps; some may have only one. The flaps may be shaped to facilitate wrapping on the curved surface of the aorta. The flaps may be wrapped in a variety of patterns and directions over the aorta. For example, the flaps may wrapped helically or non-helically over the aorta, and they may overlap one another or lie separate. The flaps may define slots or grooves to avoid wrapping or disturbing the coronary arteries. In addition, the flaps can, but need not, be affixed to the aorta by, for example, tacks, sutures, or cement. Also, the tips of the flaps may in some cases be tied or stitched together after deployment. The ring and flaps may be made from a variety of materials, such as a plastic.

FIG. 2 also shows that the ring includes detents 22 (such as tacks or clips) that can provide traction to prevent ring slippage along the aorta. Detents may be positioned all around the inner surface of the ring. Other embodiments may have no or few detents.

FIG. 3 is a plan view of another embodiment of an aortic annuloplasty ring 30. In this embodiment, the ring has a “C” shape and is sized to fit around the aortic root and engage the root circumferentially. The ring's shape may be that of a circle's arc, but it may have other overall shapes, such as a shape corresponding to a typical aortic root's outer surface. FIG. 4 is a perspective view of the embodiment of FIG. 3. The C shape defines an gap G through which the aorta passes as the ring is deployed. The ring may be deformable. Preferably, the ring is deformable enough to permit it to be manually adjusted by, e.g., pressing the ring between an operator's fingers to narrow the gap G after the ring is positioned around the aorta. The deformation should be largely nonresilient: the ring should tend to keep its new shape when it has been thus adjusted. The ring may also be so deformable as to permit the ring to be loosened by prying its ends apart with the operator's fingers.

The ring may be formed from a variety of materials. The material is preferably biocompatible so that the ring does not provoke an immune response or other adverse reaction. The material is also preferably non-biodegradable, so that the ring persists in the body until it is deliberately removed. Preferable materials include gold, silver, titanium, nickel-titanium alloy, and combinations of these. An alloy having at least 23-karat gold is preferred for its malleability, nonresilience, and consequent ease of adjustment; indeed, pure (i.e., 24-karat) gold is best in this regard. However, lesser amounts of gold may be used instead. For example, the gold may be alloyed with silver (preferably less than 10% silver). Other possible alloys are gold and titanium; gold, silver, and titanium, or other metals. Silver may provide bacteriostasis. Barium may provide radioopacity. Nickel-titanium may provide shape memory.

The material may include a thermoplastic elastomer. The shape and/or flexibility of such a material may be temperature-dependent. For example, the thermoplastic elastomer may be selected so that it is less flexible at body temperature (typically around 37° C.) than at room temperature (for example, in the range of 15° C. to 24° C.). A ring including such a material could be flexible enough to permit adjustment before it has warmed to body temperature and then could become inflexible enough at body temperature to impede further adjustment in response to blood pressure or the heart beat's force. In some embodiments, the thermoplastic elastomer may be selected so that the ring is manually deformable at a temperature below body temperature.

The material may be selected so that the ring is so rigid at body temperature as not to deform in response to arterial blood pressure (up to about 200 mm Hg), in response to repeated heart pressure cycles (up to about 160 beats per minute), or in response to motion of the heart or aortic root (from a heartbeat).

The “C” ring will typically be an arc of about 240 degrees to about 270 degrees. In other words, the gap defined by the ring will typically account for at least one fourth but usually less than one third of the ring's circumference.

When placing a “C” ring on the aorta of a particular patient, an operator typically selects a ring size that approximates or slightly exceeds the aorta's diameter. This maximizes contact between the ring and the aorta and also minimizes the adjusting required to improve leaflet coaption. Typical human aortas have diameters in the range of about 1 cm to about 3 cm, with some aortas as large as 5 cm or, rarely, larger still. Accordingly, rings will typically be made that have a major diameter D (FIG. 3) in these ranges. In some instances, a kit can be provided that includes rings having several different major diameters. The operator can measure the subject's aortic diameter and select a ring having a corresponding diameter.

The ring stiffness depends on the ring material and ring's minor diameter d (FIG. 3), i.e., its thickness. For the preferred materials, the desired ring stiffness will result from a minor diameter d in the range of about 0.1 mm to about 2 mm.

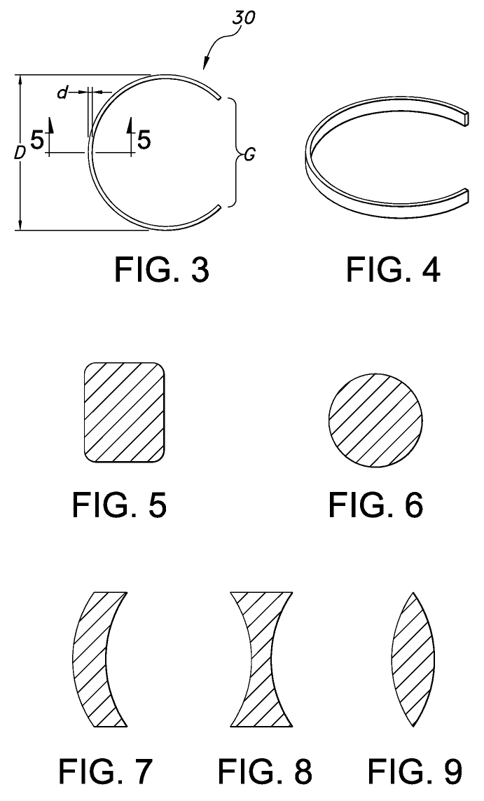

The ring may have edges. The edges are preferably rounded to prevent trauma to the surrounding tissue, particularly to the nearby coronary arteries. The edges of the ring may be slightly rounded so that a cross-section of a segment of the ring (taken, for example, at line 5-5 of FIG. 3) has rounded corners, as shown in FIG. 5. Among other possible ring cross-section shapes are the circular shape shown in FIG. 6, the convex-concave shape shown in FIG. 7, the concave-concave shape shown in FIG. 8, and the convex-convex shape shown in FIG. 9. Additionally, the ring may have different cross-sectional shapes in different regions along the length of the ring.

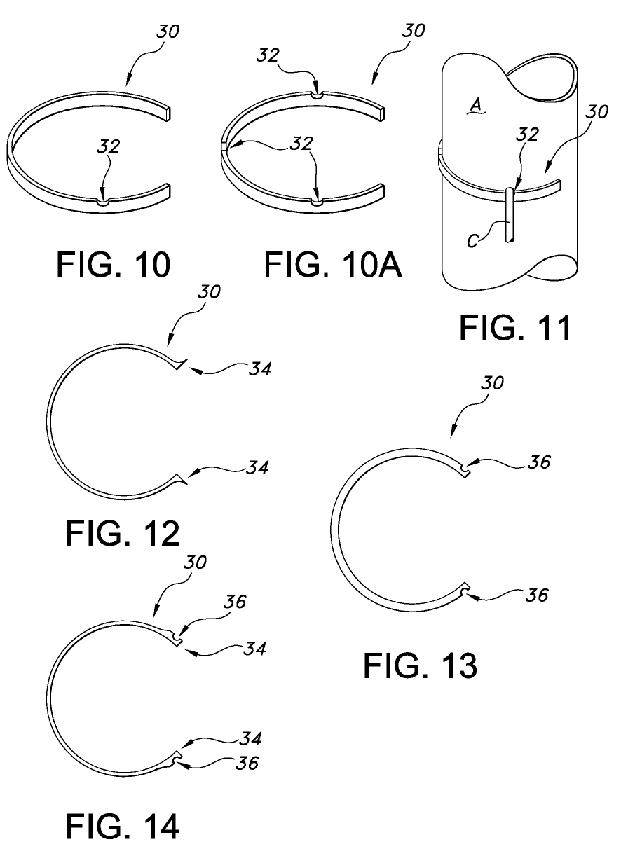

FIG. 10 shows an embodiment in which the ring defines a groove 32. The groove 32 provides a contour to fit a coronary artery so that the ring may snugly engage the aortic root without impinging the coronary artery. A groove also provides a location for tying down the ring in the subcoronary position. FIG. 10A shows an embodiment in which the ring has three grooves 32. In other embodiments, a ring may have two grooves, or more than three grooves. If a ring has multiple grooves, it is preferable to space the grooves equally around the ring to distribute forces evenly. FIG. 11 shows a side view of an aorta A having a coronary artery C branching therefrom, with a grooved ring 30 circumferentially engaging the aorta and the ring groove 32 lessening trauma to the coronary artery.

The rings described herein may be deployed in a number of ways. For example, during open thoracic surgery, the ring may be slipped around the exposed aorta. During a thoracoscopic procedure, a ring may be delivered through an endoscopic instrument and positioned using the appropriate tools. A ring may be introduced in a catheter that is advanced through the vasculature to the aorta and positioned around the aorta through an incision in the aortic wall.

Once positioned, a ring may be secured by tacking or other affixation (such as by detents 22 of FIG. 1) to the outer surface of the aorta. In addition, a ring may be affixed by devices that penetrate the full thickness of the aortic wall and are affixed on the inner surface of the aorta. For example, if access to the interior of the aorta is available (as by catheterization or by incision into the aorta), then a ring may be attached to the aorta by stitching, stapling, or riveting through the full thickness of the aorta.

Once deployed, the rings described herein may be adjusted in a variety of ways. As described above, a ring may be adjusted manually. For example, a ring as shown in FIG. 1 may be adjusted by pulling the second end 14 through the fastener 16. A ring as shown in FIG. 3 may be adjusted by squeezing the ends together or by prying them apart. Attachments or accessories may also be used to adjust a ring. For example, a clamp or wrench may be applied to a ring to squeeze or pry it. Arms of a clamp may engage respective ends of a ring. The grip of the clamp may be facilitated by providing a projection or indentation on one or both ends. FIG. 12 depicts an exemplary embodiment of a ring 30 having projections 34 on the ends. FIG. 13 depicts an exemplary ring 30 having indentations 36 on the ends. As shown in FIG. 14, one or both ends of a ring may have a combination projection/indentation 38.

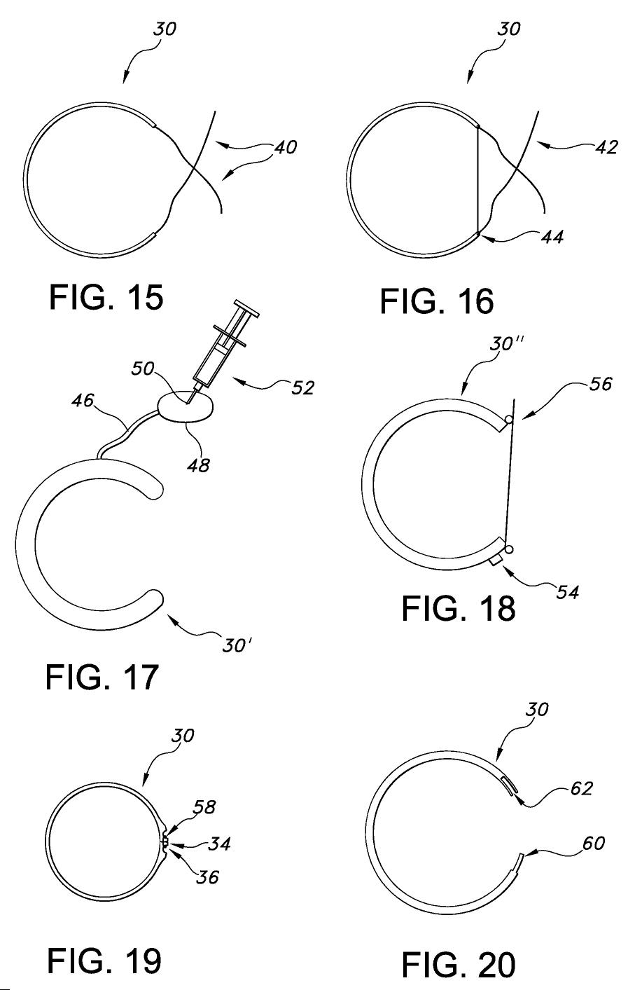

A ring may be adjusted by pulling one or more strings, sutures, guidewires, or other filaments attached to one or both ends of the ring. As shown in FIG. 15, filaments 40 may be attached to ends of a ring 30 and be pulled in opposite directions to tighten the ring. As shown in FIG. 16, a single filament 42 may be slideably coupled to at least one end of a ring 30 by a couple 44. Alternatively, a filament may be secured to one end and slideably coupled to the other, so that there is one free end which may be pulled to tighten the ring. The filaments may be removable from the ring so that they may be disconnected from the ring once the ring is adjusted. Alternatively, the filaments may remain affixed to the ring to permit further adjustment after the ring is deployed. In some cases, the loose end(s) of filament(s) may be brought out to the skin surface or just below the skin surface to facilitate the further adjustment. The filaments may disposed in conduits, such as tubes, to protect the filaments from scarring or adhesion and to enable their controlled movement by an operator.

Additional adjustment systems are contemplated. For example, as depicted schematically in FIG. 17, a ring 30′ may be an inflatable “C” cuff that fits around the aorta. In this embodiment, the ring may be adjusted by inflating the cuff. As the cuff inflates, it exerts the desired compressive force on the aorta. Alternatively, a ring may be as described earlier, with an inflatable cuff attached to the outside of ring. Inflating the cuff can exert compressive force on the ring, which deforms on response. The cuff may then be deflated, or it can be kept inflated to maintain the deformed state of the ring. In yet another alternative, a ring can be embedded in an inflatable cuff. When the cuff is inflated, it exerts compressive force on the aorta, and the embedded ring helps the cuff to keep its shape and remain in position.

The cuff may be inflatable by a liquid, a gas, or other fluid material. A line 46 may be coupled in fluid communication with the ring cuff 30′. In an embodiment, the line 46 can connect in fluid communication with a bladder 48. The bladder 48 may be disposed in a patient subcutaneously, with a port 50 accessible just beneath the skin. A source of fluid such as a syringe 52 may be applied to the port to introduce or withdraw fluid from the bladder 48, thereby inflating or deflating the ring 30′, respectively.

In yet another embodiment, depicted schematically in FIG. 18, a ring 30″ may include a controller 54 coupled to an adjustment system such an electronic fulcrum or gear arrangement 56. The controller 54 may be an RF receiver that receives commands from an external control (not shown). In response to such commands, the controller 54 may instruct the arrangement 56 to open or close the ring 30″. The controller 54 and/or arrangement 56 may also be responsive to magnetic signals.

Rings may be sealed shut to prevent undesired loosening or opening. A wide variety of sealing systems may be appropriate for this purpose. For example, the ends of a ring 30 may be glued together. Alternatively, as shown in FIG. 19, once the ends of a ring 30 are brought to the final adjustment position, the ends may be tied together by, e.g., a tie 58. (FIG. 19 shows the ring fully closed in its final adjustment position, but it need not be.) The tie 58 may fit around projections 34 of the ends. Alternatively, or simultaneously, tie 58 may fit in an indentation 36, such as a groove. In another embodiment, depicted in FIG. 20, one end of a ring 30 may have a boss 60 that fits into a receptacle 62. The boss 60 may be, for example, glued or welded into receptacle 62. The boss 60 may be so sized as to engage the receptacle 62 in friction-tight press-fit.

A ring sizer may be provided to determine the appropriate ring size to use with a particular patient. Aortic size may be difficult to determine prior to a surgery or other procedure, so a sizing system may be used during such surgery or procedure. A sizer may be a calibrated ring or strap that can be fitted around the aorta at the appropriate position, and a size read therefrom. The sizes indicated on the sizer may correspond to sizes of rings available. A kit may be provided that includes a sizer and a selection of rings of various sizes. If appropriate, the kit may also include an adjustment tool, such as a filament, a clamp, or a line/bladder system as described for FIG. 17.

During the deployment and/or adjustment of an aortic annuloplasty ring, it may be desirable to monitor blood flow through the aortic valve to determine whether the ring is appropriately adjusted. For example, blood flow through the valve may be monitored to determine whether the ring has sufficiently coapted the valve leaflets to eliminate aortic regurgitation. If blood flow is not adequately corrected, the ring may be further adjusted. If blood flow is overcorrected (for example, by creating aortic stenosis), the ring may be loosened. A number of methods may be employed for assessment of blood flow, such as echocardiography (transesophageal and/or transthoracic), intraoperative leak tests, direct observation (e.g., through a catheter camera), and fluoroscopy.

1. An aortic annuloplasty ring, comprising:
a collar having first and second ends that together form a fastener operable to secure the first and second ends together and thereby so shape the collar as to engage the aorta circumferentially; and
a flap depending from the collar for wrapping over the aorta;
the ring being sized to fit around the aorta, and further being transitionable between a first state, in which the fastener does not secure the first and second ends together, and a second state, in which the fastener so secures the first end to the second end that the collar is shaped to engage the aorta circumferentially.

2. The ring of claim 1, wherein the fastener comprises a plurality of catches on the second end selectively receivable by an aperture in the first end, thereby making the ring adjustable.

3. The ring of claim 1, wherein the collar can lie substantially flat in the first state.

4. The ring of claim 1, wherein the collar has a substantially circular shape in the second state.

5. The ring of claim 1 wherein the flap is one of a plurality thereof that depend from the collar.

6. The ring of claim 1, wherein the ring is reversibly transitionable between the first state and the second state.

7. The ring of claim 1, wherein the ring is not reversibly transitionable between the first state and the second state.

8. The ring of claim 1, wherein the fastener is adjustable.

9. The ring of claim 1, further comprising a detent disposed on the collar for engaging the aorta.

10. The ring of claim 1, further comprising a plurality of detents disposed on the collar for engaging the aorta.

11. The ring of claim 1, wherein the collar is formed at least in part from a plastic.

12. The ring of claim 1, wherein the flap is formed at least in part from a plastic.

13. The ring of claim 1, further comprising an adjustment device coupled to the collar.

14. The ring of claim 13, wherein the adjustment device comprises a filament.

15. An adjustable aortic annuloplasty ring, comprising:
a collar having first and second ends that together form a fastener operable to secure the first and second ends together and thereby so shape the collar as to engage the aorta circumferentially, the fastener having a plurality of catches on the second end selectively receivable by an aperture in the first end, thereby making the ring adjustable; and
a plurality of flaps depending from the collar for wrapping over the aorta;
the ring being sized to fit around the aorta, and further being transitionable between a first state, in which the fastener does not secure the first and second ends together, and a second state, in which the fastener so secures the first end to the second end that the collar is shaped substantially circularly to engage the aorta circumferentially.

16. An aortic annuloplasty ring, comprising:
a ring, having a “C” shape, and being so sized as to fit around and circumferentially engage an aortic root;
the ring being formed at least in part of a biocompatible material so deformable as to permit manual adjustment of the ring and so nonresilient as to keep the shape into which it is adjusted.

17. The ring of claim 16, wherein the material is gold.

18. The ring of claim 16, wherein the material is 24-karat gold.

19. The ring of claim 16, wherein the material is an alloy.

20. The ring of claim 19, wherein the alloy includes at least 23-karat gold.

21. The ring of claim 19, wherein the alloy includes gold and silver.

22. The ring of claim 21, wherein the alloy includes less than 10% silver.

23. The ring of claim 19, wherein the alloy includes gold and titanium.

24. The ring of claim 19, wherein the alloy includes gold, silver, and titanium.

25. The ring of claim 16, wherein the material is a thermoplastic elastomer.

26. The ring of claim 25, wherein the thermoplastic elastomer is less flexible at body temperature than at room temperature.

27. The ring of claim 25, wherein the material is so deformable as to permit manual adjustment of the ring at temperatures below body temperature.

28. The ring of claim 16, wherein the ring, at body temperature, is so rigid as not to deform.

29. The ring of claim 16, wherein the ring, at body temperature, is so rigid as not to deform in response to arterial blood pressure.

30. The ring of claim 16, wherein the ring, at body temperature, is so rigid as not to deform in response to motion of the aortic root.

31. The ring of claim 16, wherein the material is non-degradable.

32. The ring of claim 16, wherein the “C” shape defines a gap, and the gap accounts for at least one fourth of the circumference of the ring.

33. The ring of claim 32, wherein the gap accounts for between one fourth and one third of the circumference of the ring.

34. The ring of claim 16, wherein the “C” shape defines a gap, and the gap accounts for at most one third of the circumference of the ring.

35. The ring of claim 16, wherein the ring defines a diameter, the diameter being at least 1 centimeter.

36. The ring of claim 35, wherein the diameter is at most 5 centimeters.

37. The ring of claim 16, wherein the ring defines a diameter, the diameter being at most 5 centimeters.

38. The ring of claim 16, wherein the ring defines a diameter in the range between about 1 centimeter and about 3 centimeters.

39. The ring of claim 16, wherein the ring has rounded contours.

40. The ring of claim 39, wherein the contours of the ring are so rounded as to minimize trauma to a coronary artery.

41. The ring of claim 16, wherein the ring defines a groove so contoured as to receive a coronary artery.

42. The ring of claim 16, wherein the ring circumscribes an arc of a circle.

43. The ring of claim 16, wherein each end of the ring has a projection for engaging a respective arm of an adjustment wrench.

44. The ring of claim 16, wherein each end of the ring defines an indentation for engagement by a respective arm of an adjustment wrench.

45. The ring of claim 16, further comprising a filament coupled to each end of the ring.

46. The ring of claim 16, further comprising an inflatable cuff and a bladder in fluid communication with the cuff.

47. An aortic annuloplasty method, comprising:
disposing an aortic annuloplasty ring around an aorta, the ring including a collar having first and second ends, the first and second ends forming a fastener operable to secure the first and second ends together, the ring further including a flap depending from the collar;
fastening the first and second ends of the collar to each other, thereby so shaping the collar as to engage the aorta circumferentially; and
wrapping the flap over the aorta.

48. The method of claim 47, further comprising unfastening the first and second ends of the collar and refastening the first and second ends in a different position.

49. The method of claim 47, further comprising observing blood flow through the aortic valve and adjusting the ring in response to the blood flow.

50. The method of claim 49, wherein adjusting comprises tightening the ring in response to blood flow indicative of aortic insufficiency.

51. The method of claim 49, wherein adjusting comprises loosening the ring in response to blow flow indicative of aortic stenosis.

52. The method of claim 47, further comprising actuating an adjustment device coupled to the ring.

53. The method of claim 52, wherein the adjustment device comprises a filament, and actuating comprises pulling the filament.

54. The method of claim 52, wherein the adjustment device comprises a wrench having two arms, the arms of the wrench being engaged to the ends of the ring, and actuating comprises clasping the wrench.

55. The method of claim 52, wherein the ring further comprises an inflatable cuff, and the adjustment device comprises a bladder in fluid communication with the cuff, and wherein actuating comprises introducing fluid into or removing fluid from the bladder, thereby changing the inflation of the cuff.

56. The method of claim 47, further comprising measuring the circumference of the aorta and thereby selecting the size of the ring.

57. An aortic annuloplasty method, comprising:
disposing an aortic annuloplasty ring around an aorta root, the ring having a “C” shape, the ring being so sized as to fit around and circumferentially engage the aortic root, and the ring being formed at least in part of a biocompatible material so deformable as to permit manual adjustment of the ring and so nonresilient as to keep the shape into which it is deformed; and
deforming the ring to circumferentially engage the aortic root.

58. The method of claim 57, wherein the ring comprises a flap depending from the ring, and the method further comprises wrapping the flap over the aorta.

59. The method of claim 57, further comprising observing blood flow through the aortic valve and adjusting the ring in response to the blood flow.

60. The method of claim 59, wherein adjusting comprises tightening the ring in response to blood flow indicative of aortic insufficiency.

61. The method of claim 59, wherein adjusting comprises loosening the ring in response to blow flow indicative of aortic stenosis.

62. The method of claim 57, further comprising actuating an adjustment device coupled to the ring.

63. The method of claim 62, wherein the adjustment device comprises a filament, and actuating comprises pulling the filament.

64. The method of claim 62, wherein the adjustment device comprises a wrench having two arms, the arms of the wrench being engaged to the ends of the ring, and actuating comprises clasping the wrench.

65. The method of claim 62, wherein the ring further comprises an inflatable cuff, and the adjustment device comprises a bladder in fluid communication with the cuff, and wherein actuating comprises introducing fluid into or removing fluid from the bladder, thereby changing the inflation of the cuff.

66. The method of claim 57, further comprising measuring the circumference of the aorta and thereby selecting the size of the ring.
